SEQUENCE LISTING (10 sequences)

SEQ ID NO: 1 - DNA, 21 bp, Artificial Sequence
  Synthetic Oligonucleotide
cagcagatcagccacatcgcc

SEQ ID NO: 2 - DNA, 22 bp, Artificial Sequence
  Synthetic Oligonucleotide
tgagaaaggagacccagcagcc

SEQ ID NO: 3 - DNA, 19 bp, Artificial Sequence
  Synthetic Oligonucleotide
atgtgcgtgtgccaggacc

SEQ ID NO: 4 - DNA, 20 bp, Artificial Sequence
  Synthetic Oligonucleotide
cccttcttggtgccctccag

SEQ ID NO: 5 - DNA, 23 bp, Artificial Sequence
  Synthetic Oligonucleotide
actggagagaagccgtttcggtg

SEQ ID NO: 6 - DNA, 23 bp, Artificial Sequence
  Synthetic Oligonucleotide
tgcagccttgaaagggacacacg

SEQ ID NO: 7 - DNA, 22 bp, Artificial Sequence
  Synthetic Oligonucleotide
ggcagcgaggtagtgaagagac

SEQ ID NO: 8 - DNA, 22 bp, Artificial Sequence
  Synthetic Oligonucleotide
gaaagccgatgtggtcagccaa

SEQ ID NO: 9 - DNA, 20 bp, Artificial Sequence
  Synthetic Oligonucleotide
atggggaaggtgaaggtcgg

SEQ ID NO: 10 - DNA, 21 bp, Artificial Sequence
  Synthetic Oligonucleotide
ggagtgggtgtcgctgttgaa

(19) United States
(12) Patent Application Publication
(10) Pub. No.: US 2012/0141547 A1
(43) Pub. Date: Jun. 7, 2012

(54) METHODS OF MAKING CELL SHEETS, TISSUE SHEETS AND TISSUE ENGINEERED BLOOD VESSELS

(76) Inventors: Feng Zhao, Houghton, MI (US); Kam W. Leong, Durham, NC (US)

(21) Appl. No.: 13/262,719

(22) PCT Filed: Apr. 5, 2010
(86) PCT No.: PCT/US10/29952

§ 371 Date: Jan. 23, 2012

Related U.S. Application Data
(60) Provisional application No. 61/166,447, filed on Apr. 3, 2009.

(51) Int. Cl. A61K 35/12; C12N 5/0735; C12N 5/0775; C12N 5/077; B65H 81/06; A61K 9/00; A61P 17/00; A61P 19/08; A61P 9/00; C12N 5/071; C12N 5/0789

(52) U.S. Cl. 424/400; 435/402; 435/396; 435/366; 435/325; 424/93.7; 156/184

(57) ABSTRACT

Methods of making cell sheets, tissue sheets and tissue-engineered blood vessels are provided. The methods include culturing cells on a nanoimprinted coated scaffold under hypoxic conditions to form an aligned cell sheet and then removing the aligned cell sheet from the scaffold. The cell sheets, tissue sheets and tissue-engineered blood vessels made the methods are also disclosed. The cell sheets, tissue sheets and tissue-engineered blood vessels may be implanted in subjects to treat a variety of conditions.

### CROSS-REFERENCE TO RELATED APPLICATIONS

This application claims the benefit of U.S. Provisional Patent Application No. 61/166,447 filed Apr. 3, 2009, which is incorporated herein by reference in its entirety.

### STATEMENT REGARDING FEDERALLY SPONSORED RESEARCH

This invention was made with government support under National Institutes of Health grant number EB 003447. The government has certain rights in the invention.

### INTRODUCTION

Tissue engineering holds the prospect of producing tissues in vitro to fill the need for tissue regeneration and provide faster and more complete healing for subjects. The clinical efficacy of synthetic, allogeneic or xenogeneic engineered tissues has been limited by various problems including thrombosis, immunorejection, chronic inflammation and poor mechanical properties of the tissues after implantation. In particular, cardiovascular tissue engineering and production of small blood vessels is needed.

### SUMMARY OF THE INVENTION

In one aspect, cell sheets and methods of making cell sheets are described herein. The methods include culturing cells with an at least partially coated nanoimprinted scaffold in hypoxic conditions to form an aligned cell sheet, and removing the aligned cell sheet from the scaffold.

In another aspect, cell sheets comprising aligned cells are provided. The cells in the cell sheets are aligned such that at least 75% of the cells are aligned with the nanograting on the scaffold with an angle of less than 30° in the cell sheet. The cells in the cell sheets may be aligned such that at least 65% of the cells are aligned with the nanograting on the scaffold with an angle of less than 10° in the cell sheet.

In still another aspect, methods of making tissue sheets and tissue engineered blood vessels are provided. The tissue sheets are made by stacking cell sheets. The tissue engineered blood vessels are made by wrapping the cell or tissue sheets around a mandrel to form a tube, culturing the tube in hypoxic conditions for a period of time and then removing the mandrel. The tissue sheets and tissue engineered blood vessels are also provided.

In yet another aspect, methods of treating subjects in need of tissues or blood vessels are provided. The methods include implanting the tissue sheets and blood vessels in a subject.

### BRIEF DESCRIPTION OF THE DRAWINGS

FIG. 1 is a schematic illustration showing the method of making the cell sheets, tissue sheets and tissue-engineered blood vessels described herein.

FIGS. 2A and 2B are graphs showing typical XPS spectra of PDMS (A) and TRCC coated PDMS (B) surfaces. The N1s peak region near 400 eV in the spectrum of TRCC-PDMS (B) confirmed the immobilization of TRCC on the PDMS. FIG. 2C-E show the surface topographic morphology of PDMS (C), plasma treated PDMS (D) and TRCC-PDMS (E) as detected by AFM. P indicates the periodicity and D indicates the depth of the grating in the figure.

FIG. 3 is a set of photographs showing tissue-engineered blood vessels.

FIG. 4 is a photograph of a tissue-engineered blood vessel showing a blood vessel with an internal diameter of 2 mm and an outer diameter of 4.8 mm.

FIG. 5 is a set of photographs showing the handling and suturing characteristics of the tissue-engineered blood vessels. The top right photograph shows a tissue-engineered blood vessel in perfusion flow experiments withstanding high pressure flows.

FIG. 6A is a set of photographs showing the morphology of the nuclei, and F-actin of human mesenchymal stem cells (hMSCs) grown on a nanoimprinted scaffold at 20% O₂ (NN), a flat scaffold at 20% O₂ (NF), a nanoimprinted scaffold at 2% O₂ (HN), and a flat scaffold at 2% O₂ (HF) on day 14 of culture. The arrows indicate the direction of the nanograting on the surface.

FIG. 6B is a set of photographs showing the morphology of F-actin in hMSC layers at day 14 of culture in conditions NN, NF, HN, and HF at low magnification. Uniform cell layers formed in the two low oxygen conditions, while the NN samples displayed a patchy structure. The arrows indicate the direction of the nanograting on the surface.

FIG. 6C is a graph showing the distribution of the cell nuclear alignment angles in the four conditions tested. The percentage of aligned cells with angles of less than 10° was significantly higher in HN samples than in NN samples and the distribution of alignment angles was much narrower in the HN condition than in NN.

FIG. 6D is a graph showing the cell nuclear morphology in the four conditions tested after 14 days of culture, the nuclei of the hMSCs grown under low oxygen were more elongated than their counterparts grown under normal oxygen conditions. Values are mean±SD for six samples of each condition (*P<0.05).

FIG. 7A is a set of photographs showing the morphology of fibronectin (green) in hMSCs after 7 days culture in NN, NF, HN and HF conditions. The F-actin was counter-stained with phalloidin (red). Thin arrows indicate the direction of the nanograting and thick short arrows indicate cell clusters.

FIG. 7B is a photograph of a Western blot of fibronectin after culturing for 4 or 7 days under NN, NF, HN and HF conditions (from left to right in the photograph). β-actin served as a loading control.

FIG. 7C is a graph of the quantitative analysis of the Western blot shown in FIG. 7B. The expression of fibronectin in each sample was normalized to the expression of the endogenous control β-actin. The fibronectin secretion at days 4 and 7 was significantly higher in the two hypoxic samples (HN and HF) than in the normoxic samples (NN and NF).

FIG. 8 is a set of photographs showing the extracellular matrix formation after 14 days of culture in the four conditions (NN, NF, HN and HF). The arrows indicate the direction of the nanogratings on the surface of the scaffold. Fibronectin (FN) and laminin (Lam) were secreted equally well in all four conditions. The two normoxic cultures appeared to express collagen I (Col I) and collagen IV (Col IV) at significantly lower levels than those seen in the two low oxygen cultures.

FIG. 9A is a graph showing the percentage of proliferating cells (i.e. cells in either S or M phase of the cell cycle) over a 21 day culture period in each of the indicated conditions. The data are based on flow cytometry based propidium-iodide cell cycle analysis. The total percentage of proliferating cells was significantly lower on the two nanoimprinted scaffolds than those on the flat scaffolds at day 7. After day 7 the differences were not significant.

FIG. 9B is a set of graphs showing flow cytometry results of cell viability at days 7 (top row) and 21 (bottom row) of culture under the indicated conditions. The cell viability was assessed by flow-cytometry based Annexin V/propidium iodide apoptosis assay.

FIG. 10A is a set of photographs showing the morphology of colonies formed by cells from the four culture conditions. The cells were harvested from each of the four conditions at the indicated time point and then further cultured for 14 days at a low seeding density. Colonies were stained with crystal violet.

FIG. 10B is a graph showing the colony-forming ability of hMSCs based on the data shown in FIG. 10A. A significant difference (*P<0.05) is apparent between low and normoxic cultures starting from day 4.

FIG. 10C is a photograph of a gel showing the RNA expression levels of stem cell genes Oct-4, Rex-1 and Sox-2 in hMSCs at the indicated number of days (1, 4, 7 and 21) in the indicated culture conditions (NN, NF, HN and HF). The expression was normalized to the expression of β-actin and a human embryonic stem cell was the positive control (+). Culture under low oxygen enhanced all of the mRNA expression levels in hMSCs grown on either a nanoimprinted or flat scaffold.

FIG. 11A is a set of photographs showing increased and larger lipid droplet clusters in the cells cultured in hypoxic conditions as compared to the normoxic cells after adipocyte differentiation for 21 days.

FIG. 11B is a set of photographs showing Von Kossa staining of cells after 21 days of culture in osteoinductive media. Significant calcium deposition was displayed in the ECM in the hypoxic cultures as compared to the normoxic cultures.

### DETAILED DESCRIPTION

Methods of making cell sheets are provided herein. The methods include nanoimprinting a scaffold with nanogratings and covering the scaffold with a coating to produce an at least partially coated nanoimprinted scaffold. Cells are cultured on the nanoimprinted coated scaffold in 1% to 10% oxygen conditions to form a cell sheet. The cell sheet is then removed from the coated scaffold. The resulting cell sheets are substantially free of the scaffold. The cells in the cell sheet may be aligned with each other and with the nanograting on the scaffold such that at least 75% of the cells are aligned with an angle of less than 30°. The cells in the cell sheet may be aligned with each other and with the nanograting on the scaffold such that at least 65% of the cells are aligned with an angle of less than 10°. The cell sheets can be removed from the scaffold without the use of toxic or noxious solvents and without leaving a substantial amount of residual scaffold in or attached to the cell sheet.

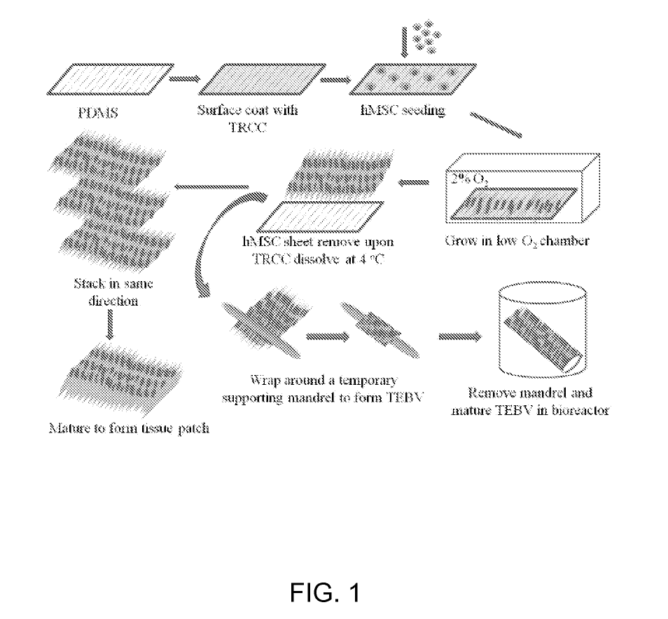

The cell sheet resulting from this process can be used in a wide variety of applications which will be apparent to those skilled in the art. The cell sheets may be used to form tissue sheets by stacking cell sheets together such that the cells in each cell sheet are aligned. The cell sheets may also be wrapped around a mandrel to form a tube and cultured in 1% to 10% oxygen to form tissue-engineered blood vessels (TEBV). A schematic illustration of the method is shown in FIG. 1.

The resultant tissue sheets and TEBV may be used to treat subjects in need of tissues by implanting the tissue sheets or TEBV into the subject. Those of skill in the art will appreciate that subjects having a wide range of clinical presentations may be in need of a tissue implant. For example, in subjects with vascular disease the TEBV may be implanted and used to replace a damaged blood vessel. Tissue sheets may also be used to treat accident victims, burn victims, subjects with skin diseases and subjects with vascular diseases.

The cells sheets are made using a scaffold for support during growth of the cells and then the scaffold is removed prior to use. The scaffold need not be biodegradable because it will not be implanted into a subject. The scaffold may be constructed from any suitable material, including but not limited to plastic, metal or ceramic. Suitably the scaffold is constructed of poly(dimethylsiloxan) (PDMS), polystyrene, poly L-lactic acid, poly glycolic acid, poly hydroxybutyrate, polycarbonate (PC), polycaprolactone (PCL), polymethylmethacrylate (PMMA), or other thermoplastic polymers or combinations thereof.

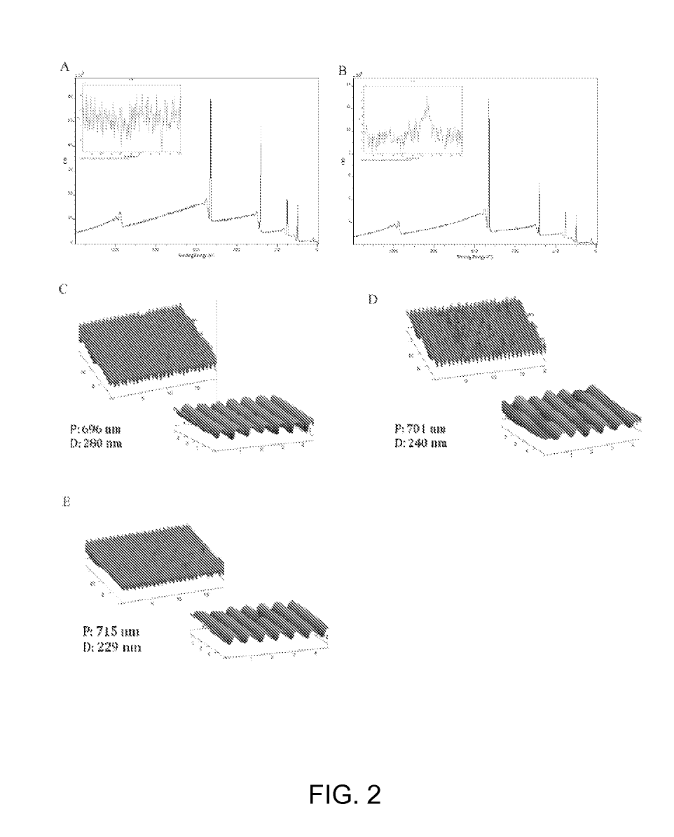

The scaffold is nanoimprinted with a pattern or a nanograting. In the Examples, a nanograte was imprinted on the surface of the scaffold using a polymethylmethacrylate coated Si master molds. The nanograte may have any suitable pattern. In the Examples, a pattern with a 280 nm depth, 350 nm width and 700 nm pitch was used. Those of skill in the art will appreciate that the nanogrates may have a wide range of depths and widths limited by the ability to produce an intact cell sheet. Nanogrates with widths up to 500 nm, 1 μm, 2 μm, 4 μm, 7 μm, 10 μm, 20 μm, 40 μm or even 80 μm and depths up to 300 nm, 400 nm, 500 nm, 1 μm, 2 μm, 4 μm, 8 μm, 10 μm, 20 μm, 30 μm, 40 μm, or 50 μm are suitable for use in the methods. Nanogrates with widths of at least 50 nm, 100 nm, 200 nm, 250 nm, or 300 nm and depths of at least 25 nm, 50 nm, 100 nm, 150 nm, 200 nm, or 250 nm are suitable for use in the methods. FIG. 2A shows typical XPS spectra of a scaffold.

The scaffold is covered with a coating. The coating may be comprised of any combinations of normal constituents of the extracellular matrix (ECM). Those of skill in the art will appreciate that a wide variety of coatings may be used, including but not limited to, chitosan, hydroxybutyl chitosan, collagen, fibronectin, laminin, elastin, fibrin, gelatin, proteoglycans, hyaluronan, or combinations thereof. In the examples, hydroxybutyl chitosan and collagen were used. FIG. 2B shows typical XPS spectra of a coated scaffold. The coated scaffolds used in the Examples had a coating about 10 nm thick. The thickness of the coating will affect the periodicity of the nanograting on the scaffold. Those skilled in the art will understand that a thicker or thinner coating may be used.

Cells are then cultured on the coated scaffold under low oxygen (i.e. hypoxic) conditions. For example, the cells may suitably be cultured in 1% to 10% oxygen, suitably 1% to 7% oxygen, suitably 1% to 5% oxygen, suitably 1% to 3% oxygen. The cells may be cultured on the scaffold in a low oxygen environment for at least 2 weeks and suitably up to 5 weeks or more. The cells will form a cell monolayer on the scaffold and will align to form a cell sheet.

Those skilled in the art will appreciate that many different types of cells may be used in the methods, including but not limited to, mesenchymal stem cells, myocyte precursor cells, myocytes, fibroblasts, chondrocytes, endothelial cells, epithelial cells, embryonic stem cells, hematopoetic stem cells, anchorage-dependent cell precursors, induced pluripotent stem cells (iPS cells), including adult fibroblasts, hMSCs, keratinocytes, and other somatic cells or combinations thereof. In the Examples, human mesenchymal stem cells (hMSCs) were used. As demonstrated in the Examples, the hMSCs were maintained in an undifferentiated, proliferative state in the cell sheets. The resultant cell sheets may be exposed to differentiation cues either prior to implantation during culturing of the cell sheets or may remain in an undifferentiated state and receive localized cues to aid proper differentiation after implantation into a subject. The hMSCs may be stimulated to differentiate along neuronal, myogenic or osteogenic lines.

In forming the cell sheets, the cells produce extracellular matrix (ECM) and form tight junctions with neighboring cells to allow cell-cell communication as demonstrated in the examples. The cellular organization of tissues provides functional competence to many tissue types. In many cases cellular organization requires alignment of the cells. In the cell and tissue sheets made by the methods described here, the cells are aligned. Suitably, at least 75%, 80%, 85%, 90%, 95%, 98%, 99% or 100% of the cells in the aligned cell sheet are aligned with an angle of less than 30°. Suitably at least 60%, 65%, 70% or 75% of the cells in the aligned cell sheet are aligned with an angle of less than 10°.

The cell sheets are then removed from the coated scaffold to form a substantially scaffold-free cell sheet. The cell sheet may be removed by gently peeling the cell sheet off of the scaffold. Alternatively, the scaffold may be coated with a coating containing a temperature sensitive polymer, such as hydroxybutyl chitosan used in the Examples. By reducing the temperature to 4° C., the hydroxylbutyl chitosan dissolves and the cell sheet can be removed from the scaffold. Those of skill in the art will appreciate that other coatings capable of being dissolved using non-toxic means may be used to coat the scaffold.

The cell sheets may be made in any size or configuration desired. For example, the cell sheets may be circular, rectangular, square or any other shape. In the Examples, rectangular and circular cell sheets were made. The cell sheets may also be made in any size. For example, the tissue sheet could be 1 cm×1 cm to 20 cm×20 cm. In Example 1, a 6 cm×5 cm rectangular PDMS was used to make the TEBV. In Example 2, a 1.6 cm diameter circular cell sheet was made.

The cell sheets may be used to make tissue sheets by stacking a plurality of the cells sheets on top of each other. Suitably the cell sheets are stacked with the cells oriented in the same direction to form tissue sheets. Suitably at least 4, 5, 6, 8, 10 or 12 cell sheets are stacked to form a tissue sheet. Suitably not more than 15, 18, 20, 25 or 30 cell sheets are stacked to form a tissue sheet. The tissue sheets are cultured for at least 2 weeks in low oxygen to allow the cell sheets to form a tissue sheet. The resulting tissue sheets may be made in any size or configuration desired. For example, the tissue sheet could be 1 cm×1 cm to 20 cm×20 cm.

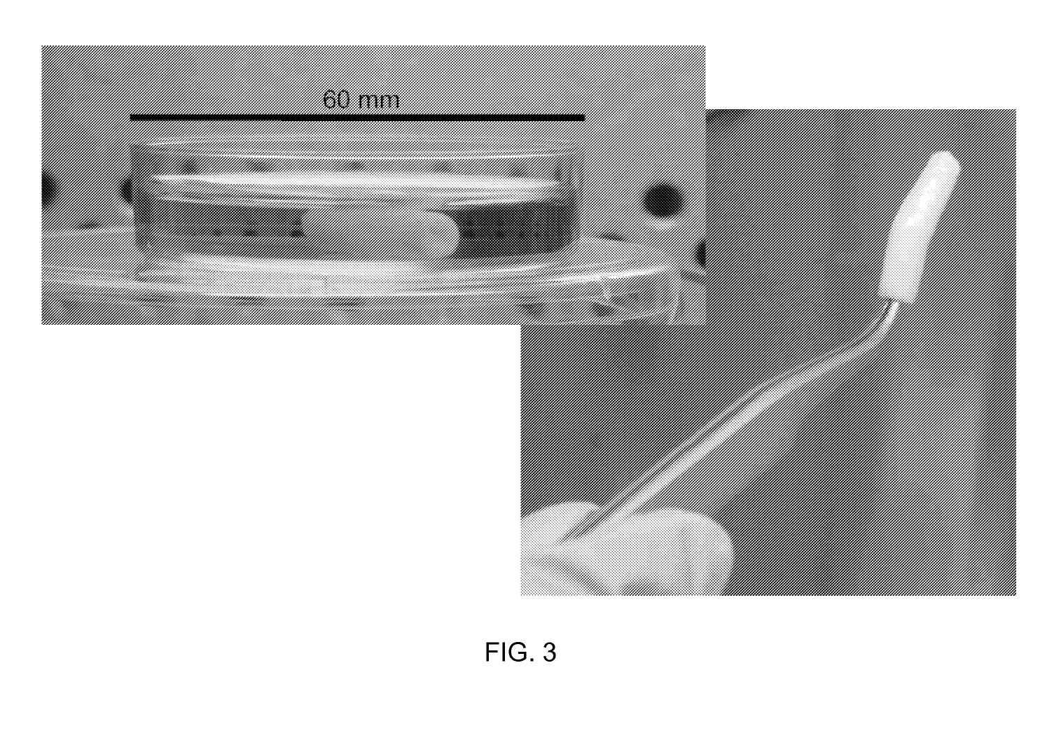

Alternatively, the cell sheets may be used to form TEBV by wrapping a plurality of cell sheets around a mandrel to form a tube. Suitably at least 4, 5, 6, 8, 10 or 12 cell sheets are wrapped around the mandrel to form a TEBV. Suitably not more than 15, 18, 20, 25 or 30 cell sheets are wrapped around the mandrel to form a TEBV. The mandrel may be any suitable diameter such that the inner diameter of the resulting TEBV may be between about 0.5 mm and about 6 mm. For example, TEBV with inner diameters of at least about 0.5 mm, 0.75 mm, 0.9 mm, 1 mm, 1.5 mm, 2 mm, 3 mm, 4 mm, 5 mm or 6 mm may be useful in various applications. Those of skill in the art will appreciate that the required inner diameter of the TEBV will be determined based upon the end use in a subject. The tube is then cultured in low oxygen for at least one week, suitably two weeks prior to removing the mandrel to form the TEBV. Suitably the oxygen concentration is between 1% and 5%, suitably 1% to 3%. The TEBV will then be matured in a bioreactor for up to 2 months. A resultant TEBV is shown in FIG. 3. The TEBV are easily handled and can be sutured into place readily. In addition, the TEBV can be attached to a bioreactor as described in the examples and in perfusion flow experiments has withstood high pressure flow conditions.

The cell sheets, tissue sheets and TEBV made by the methods disclosed herein may be used for a wide variety of purposes readily apparent to those of skill in the art. The ultimate use of the cell sheet will be important in determining the cells used to make the cell sheets. The tissue sheets may be used to make many different tissues including, but not limited to, skin, bone, muscle tissue or even nerve tissue. The cell sheets, tissue sheets and TEBV may be implanted in a subject. The subject may be any mammal, including humans. The cell sheets, tissue sheets and TEBV may be implanted by surgery or by injection in the area in need of treatment. For example, tissue sheets used to replace damaged skin may be implanted in the skin at the site of damage. Tissue sheets may also be injected in a site in need of bone regeneration to regenerate bone tissue. In another embodiment, a TEBV may be used to replace or repair a blood vessel in a subject during a surgical procedure. The Examples below are meant to be illustrative and not to limit the scope of the invention. Each and every reference cited herein is hereby incorporated by reference in its entirety.

### EXAMPLES

### Example 1

### Development of Aligned Cell Sheets and Blood Vessels Production of Nanograting and Surface Coating on PDMS Surface

The nanopattern was produced on poly(dimethylsiloxan) (PDMS) using soft lithography on a nanoimprinted poly(methyl methacrylate) (PMMA)-coated Si master mold. The gratings on the nanoimprinted PMMA master molds were 280 nm in depth, 350 nm width and had a 700 nm periodicity. The patterned and nonpatterned PDMS were plasma treated at 10 W for 10 s, and immersed into a cooled solution of thermoresponsive chitosan and bovine collagen I (BD Biosciences) at 4° C. for 30 min. The PDMS samples were then rinsed with dIH₂O and dried in air at room temperature. Before cell culture, the samples were sterilized with 70% ethanol for 30 min and rinsed with sterilized dIH₂O.

### Surface Characterization

X-ray photoelectron spectroscopy (XPS) spectra were obtained using a Kratos Ultra XPS system (Kratos Analytical Ltd., Surface Analysis Product Group, England). Static contact angles were measured with a Goniometer 110-00115 (Rame-hart, Inc. Mountain Lakes, N.J.). The measurements were carried out at room temperature in air with deionized water as the probe liquid. 25 μL liquid droplets were deposited onto the sample surface through a 22-gauge dispensing needle at a rate of 5 μL/sec. Each contact angle reported is an average of at least five measurements. The morphologies of the PDMS surfaces were observed by Atomic Force Microscopy (AFM) (Veeco D3100).

### Human Mesenchymal Stem Cell Culture and Cell Sheet Removal

HMSCs were cultured and expanded in α-MEM medium with 20% FBS and 1% penicillin/streptomycin (Life Technologies, Rockville, Md.) at 37° C. and 5% CO₂. The cells were seeded on the nanopatterned or unpatterned (flat) PDMS at 5×10³ cell/cm², and allowed to proliferate for 21 days in chambers that were flushed with humidified gas mixtures with the following composition: 2% O₂, 5% CO₂, 93% N₂. Normoxic cultures were used as controls, in which hMSCs seeded on the nanopatterned or unpatterned PDMS at 5×10³ cell/cm² were cultured at 95% air (20% O₂)-5% CO₂ for 21 days. The samples were immersed in culture medium at 4° C. for 30 min to dissolve the thermoresponsive chitosan. The hMSC sheets were removed from the PDMS surface after the dissolution of thermoresponsive chitosan.

### Tissue Sheet Fabrication

The cell sheets were stacked on each other with the cells orientated in the same direction, and matured in culture medium for 28 days to obtain tissue sheets. The tissue sheets are made by stacking from 5-20 cells sheets on each other. The tissue sheets may then be implanted in a tissue or used to make engineered blood vessels.

### Vascular Graft Fabrication

The cell sheets were wrapped around a temporary supporting mandrel with diameter of 2 mm. The formed vessels were grown in culture medium for 14 days under the two different O₂ tensions, and then removed from the mandrel, resulting in a tubular structure with circumferentially aligned hMSCs. The grafts were further matured in culture medium for 2 months before implantation. The TEBV were sutured into a perfusion bioreactor used to model in vivo flow conditions in blood vessels and to further mature the TEBV.

### Platelet Adhesion Assay

Whole blood was gently pipetted onto cell sheet surfaces. After incubation for 30 min at 37° C., the cell sheets were rinsed with PBS three times, and fixed in 2.5% (wt) glutaraldehyde in 0.1 M sodium cacodylate buffer (Electron Microscopy Science, Fort Washington, Pa.) at pH 7.4 for 2-4 h. The cells were then rinsed in buffer solution and dried using a series of graded ethanol wash steps, followed by critical point drying. The dried samples were sputter coated using palladium with gold plating and then observed using a scanning electron microscope (SEM) (FEI XL30).

### Immunocytochemistry Staining

Cells grown on Gel/NMCS-PLA and unmodified PLA samples were washed with PBS and fixed with 4% paraformaldehyde for 30 min, permeated with 1.0% Triton X-100, and then blocked with 10% normal goat serum. Next, the cells were incubated with the primary antibody against collagen I, collagen IV, fibronectin, laminin at a concentration of 1% overnight at 4° C. Cells were blocked using PBS with 1% BSA and incubated with by a mixture of secondary antibodies conjugated to Alexa Fluor 488 and phalloidin conjugated to Alexa Fluor 594 (Molecular Probes, Eugene, Oreg.) (Jackson Laboratories, West Grove, Pa.) stored in TBS and viewed using a Nikon Elipse (TE2000-U) fluorescent microscope.

### Statistical Analysis

Experimental results were expressed as means±standard deviation (SD). Statistical comparisons were performed by ANOVA for multiple comparisons, and statistical significance was accepted at p<0.05.

### Results and Discussion

PDMS Surface Coating with Thermoresponsive Chitosan and Collagen (TRCC) Complex

In order to endow PDMS surfaces with a temperature-responsive property and improve its cell adhesion ability, a polyelectronic complex (PEC) of collagen I and the thermally responsive polymer, hydroxybutyl chitosan (HBC), was coated on the PDMS surfaces (hereinafter called a TRCC coating). HBC is derived from chitosan, a natural abundant biopolymer with a polysaccharide backbone. HBC is synthesized by conjugation of hydroxybutyl groups to the hydroxyl and amino reactive sites of chitosan without compromising its biological properties. The modification confers chitosan with water soluble and temperature-responsive properties (Dang et al., Temperature-responsive hydroxybutyl chitosan for the culture of mesenchymal stem cells and intervertebral disk cells. BIOMATERIALS (2006) 27: 406-418). HBC used in this study dissolves at a temperature below its lower critical solution temperature (LCST) of 17° C. To facilitate the surface coating, the PDMS surfaces were subjected to O₂ plasma treatment to improve surface hydrophilicity by oxidizing the Si—CH₃ into Si—OH groups, and subsequently immersed into the TRCC solution which was cooled to 4° C. This hydrophilicity enhancement of the PDMS surface after the plasma treatment enforced the spreading and interaction between the polymers, giving rise to a more uniform polymer coating on the PDMS surfaces.

FIG. 2A shows typical XPS spectra of PDMS and TRCC-PDMS surfaces with enlargements of the N1s peak region near 400 eV in the TRCC-PDMS spectra. The N1s peak is absent in the spectrum of bare PDMS, confirming the successful immobilization of TRCC complex on PDMS. Table I shows the water contact angles of flat PDMS and TRCC-PDMS surfaces. The increase of surface wettability further proves the chemical binding of the TRCC complex on the relatively hydrophobic PDMS surface. This result demonstrates that the polymer film directly tunes the interfacial properties of PDMS surfaces. This agrees well with the XPS results. Topographic morphology detected by AFM shows that the grating depth decreased from 280 nm to 240 nm after the plasma treatment (FIG. 2B). After coating the surfaces with the complex of thermally responsive chitosan and collagen I (TRCC), the grating depth further decreased to 230 nm, suggesting a 10 nm ultra thin polymer coating on the PDMS surface. While the depth varied with surface treatment, the periodicity of the gratings remained unchanged after plasma treatment, and increased from 700 nm to 715 after surface coating with TRCC.

TABLE I
Water contact angle (°) of flat PDMS and TRCC-PDMS surfaces

| | | Plasma-treated | TRCC coated |
|---|---|---|---|
| Time (h) | PDMS | PDMS | PDMS |
| 1 | 91 ± 1 | 33 ± 2 | 11 ± 1 |
| 72 | 91 ± 1 | 68 ± 2 | 14 ± 2 |

Aligned hMSC Sheet

A uniformly aligned hMSC layer was formed on the nanopatterned PDMS surface after a 14-day culture period under 2% O₂ tension. The hMSCs were less organized at normal O₂ condition (20%). They aligned in the microscopic scale, but grew in different directions in the macroscopic scale, similar to those grown on flat PDMS surfaces at both normal and low O₂ tension. Higher amounts of ECM proteins were secreted in low O₂ samples of both nano-patterned and flat PDMS surfaces. The hMSC sheet was removed from the PDMS scaffold after the samples were immersed into tissue culture medium which was cooled to 4° C. At this temperature the HBC coating dissolved, leaving a polymer-free cell sheet. The elongated cytoskeletal structure and cell-cell interaction were well preserved without any destruction.

### Platelet Adhesion

Platelet adhesion is an important indication of thrombogenesis on material surfaces. The platelet adhesion on surfaces of the hMSC sheets was observed by SEM. No platelets and protein clusters were found on any of the hMSC sheet surfaces from all the culture conditions, suggesting significant suppression of platelet adhesion of the sheets. This is attributed to the unique antithrombogenic property of hMSCs.

### Blood Vessel Fabrication

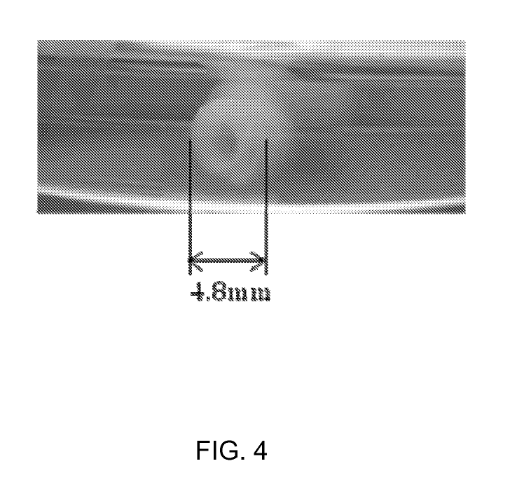

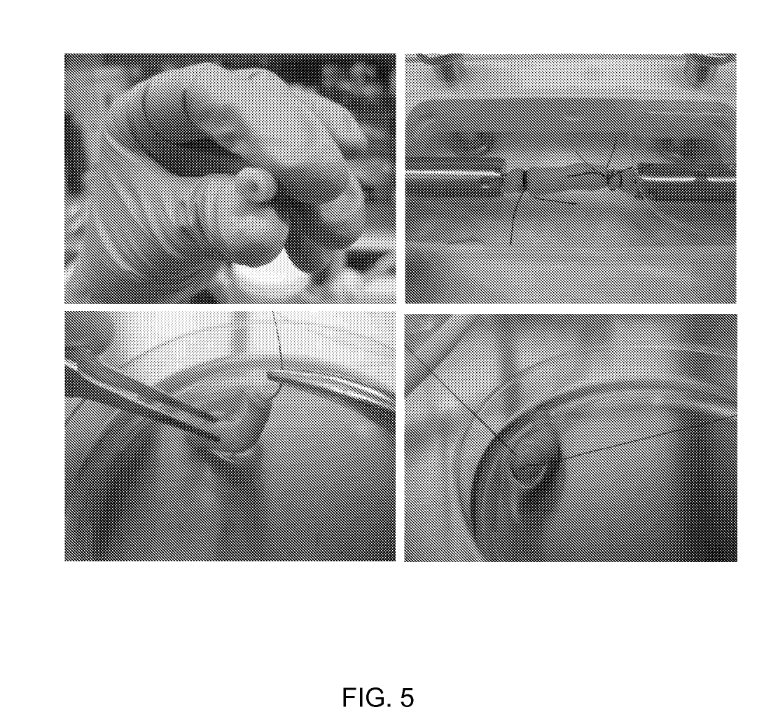

The cell sheets were wrapped around a temporary supporting mandrel and grown in culture medium for 14 days. The graft was then removed from the mandrel, resulting in a tubular structure with circumferentially aligned hMSCs. The grafts were further matured in culture medium for 2 months before implantation. FIGS. 3 and 4 show the typical tubular structure of a TEVB fabricated from the aligned hMSC sheets. The size of the graft is 5 mm in diameter and 20 mm in length. The inner diameter of the TEVB can be controlled by using a supporting mandrel with the desired diameter. FIG. 4 shows a mature TEBV in culture medium. The internal diameter of the TEBV is 2 mm and the outer diameter is 4.8 mm. FIG. 5 shows the handling and suturing characteristics of the TEBV. As shown in FIG. 5 (upper right photo) the TEBV can be attached to a bioreactor for flow experiments and was able to withstand high pressures, similar to those encountered in subjects.

### Example 2

### Maintenance of Stemness of Human Mesenchymal Stem Cells on Synthetic Nanostructures

### Materials and Methods

Production of Nanopatterned PDMS with Soft Lithography

The nanopattern was generated by using electron beam lithography on the poly(methyl methacrylate) (PMMA)-coated Si wafer as previously described, and then replicated on poly(dimethylsiloxan) (PDMS) (Ellsworth Adhesives, Germantown, Wis.) using soft lithography. Yim, et al. (2005) Nanopattern-induced changes in morphology and motility of smooth muscle cells. Biomaterials 26: 5405-5413 and Yim, et al. (2007) Synthetic nanostructures inducing differentiation of human mesenchymal stem cells into neuronal lineage. Exp Cell Res 313: 1820-1829. The gratings on the patterned PDMS surfaces were 250 nm in depth, 350 nm in width and with a 700 nm pitch. The 3D structure was imaged by atomic force microscope (Digital Instruments Dimension 3100). See FIG. 2. Before cell culture, the PDMS samples were coated with bovine collagen I (BD Biosciences, San Jose, Calif.) at 20 μg/cm², cut into disks with diameters of 1.6 cm, and sterilized using 70% ethanol.

### Cell Culture

Bone marrow-derived hMSCs were provided by Tulane University Health Sciences Center. Briefly, bone marrow aspirates of about 2 ml were drawn from healthy donors ranging in age from 19 to 49 years under an Institutional Review Board-approved protocol. Plastic adherent nucleated cells were separated from the aspirate and cultured using complete media [α-MEM with 20% FBS and 1% penicillin/streptomycin (Life Technologies, Rockville, Md.)] at 37 C and 5% CO₂. Grayson, et al. (2007) Hypoxia enhances proliferation and tissue formation of human mesenchymal stem cells. Biochem Biophys Res Commun 358: 948-953; Zhao and Ma (2005) Perfusion bioreactor system for human mesenchymal stem cell tissue engineering: Dynamic cell seeding and construct development. Biotechnol Bioeng 91: 482-493; and Zhao, et al. (2005) Effects of oxygen transport on 3-D human mesenchymal stem cell metabolic activity in perfusion and static cultures: Experiments and mathematical model. Biotechnol Prog 21: 1269-1280. The 5-6^{th} passage of hMSCs from up to 3 donors were used. The cells were cultured at 95% air (20% O₂)-5% CO₂. For hypoxia studies, hMSCs were cultured in chambers that were flushed with humidified gas mixtures of composition 2% O₂-5% CO₂-93% N₂. The cultures from the four conditions were abbreviated as: NN: 20% O₂, normoxic, nanopatterned surface; NF: 20% O₂, normoxic, flat surface; HN: 2% O₂, hypoxic, nanopatterned surface; HF: 2% O₂, hypoxic, flat surface.

### Immunocytochemistry Staining

The expression of various extracellular matrix (ECM) proteins as well as connexin-43 was examined by immunocytochemistry staining, following a previously published protocol. Grayson, et al. (2007) Hypoxia enhances proliferation and tissue formation of human mesenchymal stem cells. Biochem Biophys Res Commun 358: 948-953; and Zhao, et al. (2005) Effects of oxygen transport on 3-D human mesenchymal stem cell metabolic activity in perfusion and static cultures: Experiments and mathematical model. Biotechnol Prog 21: 1269-1280. Briefly, hMSCs grown on both PDMS surfaces were washed with DPBS and fixed with 4% paraformaldehyde for 15 min, blocked, and incubated with the primary antibody against fibronectin/collagen I/collagen IV/laminin/paxillin (Abcam, Cambridge, Mass.) or connexin-43 (Sigma, St. Louis, Mo.) for 1 h at 37° C. Cells were washed and incubated with a mixture of FITC-conjugated secondary antibodies conjugated to Alexa Fluor 488 (Abcam) and phalloidin conjugated to Alexa Fluor 568 (Jackson Laboratories, West Grove, Pa.). The samples were then washed and incubated in DAPI (Sigma) solution to counter stain the cell nuclei. Finally the samples were mounted and viewed using a Zeiss 510 confocal microscope.

### Cell Nuclei Analysis

Several separate regions of each sample were photographed using a Zeiss 510 confocal microscope with a 40× magnification. The images were analyzed with ImageJ NIH image processing software (Bethesda, Md.). Briefly, a threshold overlay was added to the cell nuclei, and then they were analyzed. Parameters such as the area and the perimeter, including major and minor axes, were processed. The cell alignment was calculated by taking the angle between the major axis of each cell nucleus and the main direction of the nanogratings. Cells were considered aligned if this angle was less than 15°. The elongation (E) parameter describes the extent to which the cell nucleus is lengthened or stretched out. It was calculated as the ratio of the long axis over the short axis minus one. The nuclear roundness (RN) parameter describes the irregularity of an object compared to a circle. Andersson, et al. (2003) Nanoscale features influence epithelial cell morphology and cytokine production. Biomaterials 24: 3427-3436. Area (A) and perimeter (P) of the filled region that describes the projected cell nuclear were used to calculate RN, RN=P/[(4πA)^{0.5}]. RN equals to 1 for a circle, and RN>1 indicates the nuclear shape is less round. The percentage of cell alignment, the E-factor, and RN were measured. For each type of condition, an average of 300 cells was counted.

### Colony Forming Unit—Fibroblast (CFU-F)

To determine CFU-F numbers, hMSCs were harvested aseptically from PDMS discs using a solution containing 0.5% trypsin/0.25% collagenase/1 mM EDTA in PBS. Cells were filtered using a cell strainer to ensure cell separation, and 800 cells were plated into a 10 cm Petri dish (14 cells/cm²). Samples for each condition were done in duplicate. The cells were grown for 12˜14 days at 37° C. and 5% CO₂ in a humidified incubator. Upon harvesting, cells were washed with PBS and stained with a 0.5% crystal violet solution for 10˜15 minutes at room temperature. Cells were washed twice with PBS and imaged with a digital camera. The visible, intensely stained colonies were counted. Triplicate matrices from each chamber were used for each data point, and two independent runs were repeated under identical conditions.

### Western Blot Analysis

The samples were removed from media and washed twice in PBS solution. Total protein was extracted from the matrices in a lysing buffer (Sigma) containing 1% Triton X-100 and protease inhibitors (Sigma). Cell lysate samples were separated by SDS gel polyacrylamide electrophoresis, and transferred electrophoretically onto nitrocellulose membranes (0.2 mm). The membranes were then blocked using a Tris buffer containing 0.1% Tween-20 and 5% dry milk. Membranes were then incubated with primary antibody overnight at 4° C., washed with blocking buffer and incubated with a horseradish peroxidase-conjugated secondary antibody for 1 hour at room temperature. Protein bands were determined by reacting the secondary antibody with an ECL plus substrate (GE Healthcare/Amersham, Pittsburgh, Pa.) to provide a chemiluminscent signal, which was detected using a Fluor Chem Imaging System (Alpha Innotech, San Leandro, Calif.). Blots were read on a densitometer and normalized to β-actin contents to quantify relative amounts of proteins. The results were reported as the ratio (density of the target band:density of β-actin band) for quantitative comparisons.

### Reverse Transcriptase-Polymerase Chain Reaction (RT-PCR)

The mRNA was extracted from 150 μl of original samples using the RNeasy MiniKit (QIAGEN, Valencia, Calif.). The purified mRNA was amplified in a one step RT-PCR reaction (QIAGEN) following the manufacturer's recommendations. Briefly, 5 μl ultrapure water containing 200 ng RNA was added to the RT-PCR mixture. This mixture contains 1 QIAGEN OneStep RT PCR enzyme mix, 5 μl of 5× QIAGEN OneStep RT-PCR buffer, 1 μl dNTP solution, 0.2 μl forward and reverse primer solutions, and 12.6 μl ultrapure water, that gives a final volume of 25 μl. The thermo cycler conditions used were: 50° C. for 30 min for reverse transcription, 95° C. for 15 min for the activation of the HotStart DNA polymerase. For the PCR we used a specific number of cycles (depending on the gene marker) consisting of the following cycles: 94° C. for 1 min, an annealing temperature depending on the gene of interest for 30 seconds, 72° C. for 1 min, followed by an extension period of 10 min at 72° C. For RNA extraction and RT-PCR procedures, we included sufficient number of negative controls (using double distilled water). These negative controls did not show positive results, which indicated the absence of cross-contamination. PCR-products were run on a 2% agarose gel, stained with ethidium bromide, and visualized under UV-light using a Fluor Chem Imaging System (Alpha Innotech). The DNA bands were quantified using AlphaEaseFC (Alpha Innotech). Intensities were normalized to β-actin. The primer sequences for target genes are listed in Table II.

TABLE II
Primer sequences for target genes

| | | Genebank | SEQ ID |
|---|---|---|---|
| Gene | Primer Sequence | No. | NO: |
| Oct-4 | F: 5′CAGCAGATCAGCCACATCGCC3′ | NM_002701 | 1 |
| | R: 5′TGAGAAAGGAGACCCAGCAGCC3′ | | 2 |
| Osteonectin | F: 5′ATGTGCGTGTGCCAGGACC3′ | NM_003118 | 3 |
| | R: 5′CCCTTCTTGGTGCCCTCCAG3′ | | 4 |
| Rex1 | F: 5′ACTGGAGAGAAGCCGTTTCGGTG3′ | NM_174900 | 5 |
| | R: 5′TGCAGCCTTGAAAGGGACACACG3′ | | 6 |
| Osteocalcin | F: 5′GGCAGCGAGGTAGTGAAGAGAC3′ | NM_199173 | 7 |
| | R: 5′GAAAGCCGATGTGGTCAGCCAA3′ | | 8 |
| GAPDH | F: 5′ATGGGGAAGGTGAAGGTCGG3′ | NT_6181 | 9 |
| | R: 5′GGAGTGGGTGTCGCTGTTGAA3′ | | 10 |

### Flow Cytometry

Flow cytometry was used for both cell cycle and apoptosis assays. Apoptosis assays were performed using an Annexin V-fluorescein isothiocyanate (FITC) apoptosis antibody (BD Biosciences) following the manufacturer's protocol. Briefly, hMSCs were trypsinized from the PDMS discs, counted, and resuspended in a binding buffer. The cell suspension was incubated with Annexin-V and propidium iodide (PI) for 15 minutes in the dark, and analyzed using a FAC Scan flow cytometer. For cell cycle assay, hMSCs were trypsinized, collected, and fixed with ice-cold absolute ethanol at 20° C. for 20 hours. The cells were then centrifuged and resuspended in a phosphate-buffered saline (PBS) buffer containing 20 μg/mL RNAase and 50 μg/mL PI. The samples were analyzed by flow cytometry within 72 hours. A peak-fitting program was used to model each of the cell cycle phases by gating analysis of the cytometric data. Statistical analysis of each iteration was performed to improve the data accuracy.

### Differentiation Assays

Differentiation assays were carried out after a 21-day culture period to determine whether the cell populations from both normal and low oxygen conditions still retained their multi-lineage potential. Both groups were differentiated under the same oxygen conditions (20% O₂). For osteogenesis, the samples were cultured in osteo-inductive media (Invitrogen, Carlsbad, Calif.). After 3 weeks, cells were fixed in 4% formaldehyde, placed in 5% silver nitrate solution, and exposed to UV light for von Kossa assays. The samples were then incubated in sodium thiosulfate solution, mounted on a microscope slide, and viewed with a Nikkon microscope.

To determine the adipogenic potential, cells were cultured with adipogenic induction medium for 21 days (Invitrogen). Adipocyte lipid-vacuole formation was detected using Nile Red staining. The cells were fixed in 4% glutaraldehyde. Stock Nile Red solution (1 mg/mL in acetone) was diluted in PBS at a ratio of 1:100 and the cells were incubated for 30 minutes. Cells were washed with PBS, mounted on a microscope slide, and viewed using a Nikkon microscope.

### Statistics/Data Analysis

Experimental results were expressed as means±standard deviation (SD) of the means of samples. All the collected data were analyzed by ANOVA for multiple comparisons, and statistical significance was accepted at p<0.05.

### Results

### Cell Alignment and Nuclear Morphology

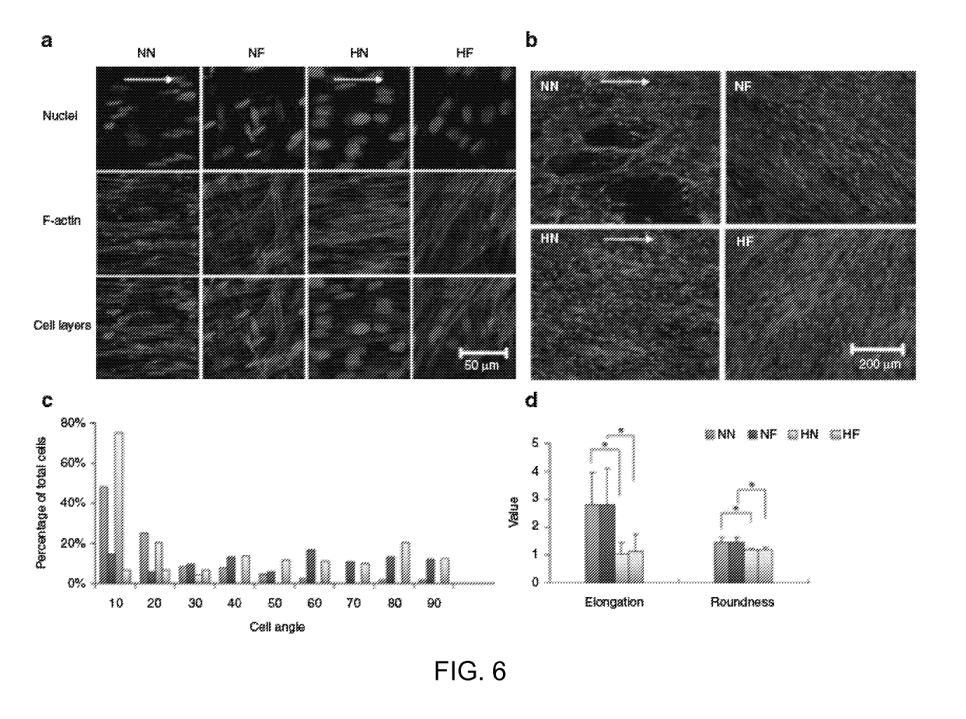

The cell layer uniformity and cell alignment on different surfaces were examined by F-actin and nuclear staining at day 14. The four groups studied are denoted by two letters: the first letter H or N represents either hypoxic or normoxic condition, and the second N or F represents nanopatterned or flat surface. The cells displayed even and fine F-actin fibers in the HN sample, while forming fiber bundles in the NF samples (FIG. 6A). A lower magnification (FIG. 6B) showed clearly a higher uniformity of the cell layer formed at the two 2% O₂ samples (HN and HF). The cytoskeleton of hMSCs was more regularly aligned along the grating axis on the HN samples than their counterparts cultured at NN condition, which demonstrated a patchy pattern. Cell alignment was defined as the angle between the major axis of each cell nucleus and the main direction of the grating axis. The percentage of aligned cells with angles less than 10° was 75% in HN samples, and 58% in NN samples (FIG. 6C). The distribution of alignment angles was also narrower in the HN condition than the NN condition. The alignment angles of HN samples were confined within 30° whereas only ˜80% in the NN samples fell within this range and the rest distributed in higher angles up to 90°. Cells cultured on flat surfaces under both normoxic and hypoxic conditions showed no directionality as expected (NF and HF).

After 14 days in culture, cells grown under low oxygen conditions (HN and HF) exhibited a nuclear morphology that was slightly elongated (FIG. 6A), with no significant difference in roundness (RN) (p>0.05) and elongation factor E (p>0.05) between the nanopatterned (HN) and flat (HF) surfaces (FIG. 6D). In contrast, cells grown at the normoxic condition displayed a more elongated nuclear morphology than their low oxygen counterparts. The nuclear factor E of hMSCs from NN condition was 2.7 compared to 1.0 for HN (p<0.01) (FIG. 6D). (A spherical nucleus would have an E=0 and RN=1; RN>1 indicates a less than spherical shape). The RN of hMSCs from normoxic conditions was both significantly higher than those from hypoxic conditions (p<0.01), indicating a less rounded cell nuclear shape. In contrast, no significant differences were observed between the nanopatterned (NN) and flat (NF) surfaces under normoxic condition (FIG. 6D).

### Gap Junction Communication and ECM Secretion

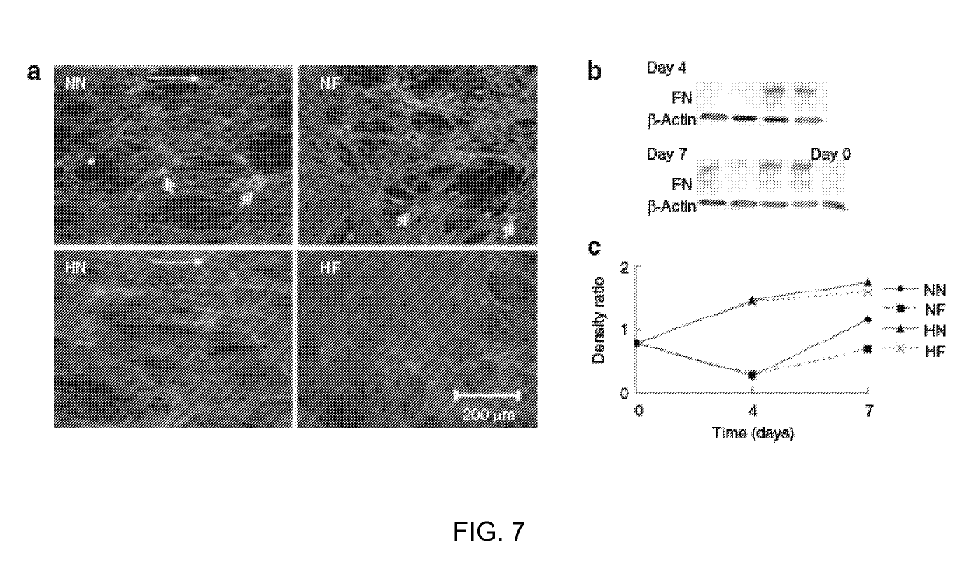

The connexin-43 secreted by cells cultured in conventional normoxic condition formed clusters on both flat (NF) and nanopatterned (NN) surfaces, whereas only distinct, small spots were observed in the 2% O₂ conditions. Fibronectin, an important ECM protein responsible for cell adhesion was examined at the early culture period. Immunofluorescent staining at day 7 showed that more abundant fibronectin was secreted in the two hypoxic samples than in the two normoxic samples (FIG. 7A). The counterstaining of F-actin illustrated that large gaps were formed between the cells on the two normoxic surfaces (NN and NF). Western blotting confirmed that the fibronectin secreted at days 4 and 7 was significantly higher in the two hypoxic samples (HN and HF) than in the two normoxic samples (NN and NF) (FIGS. 7B and 7C).

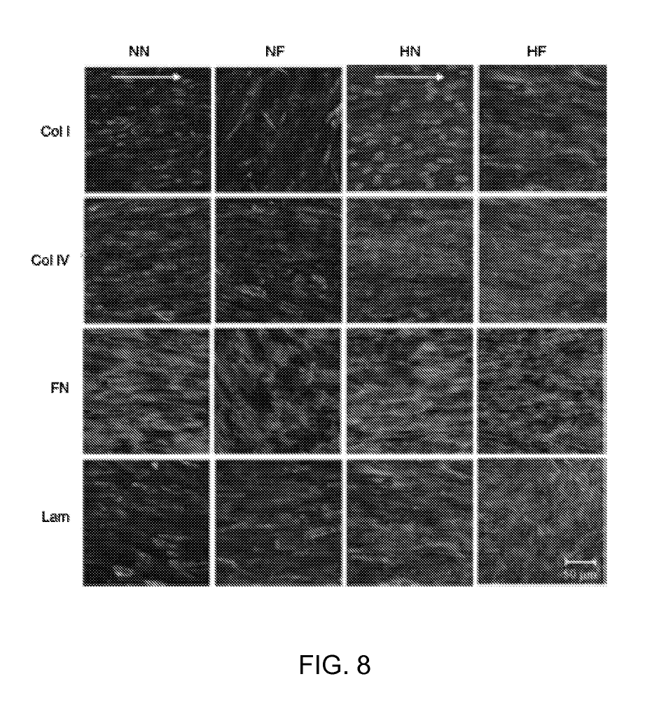

At day 14, there were significant differences in the amount of collagen I detected among the samples. The collagen I sparsely distributed in the NN and NF cultures, but organized into a network in the HN and HF samples. The two normoxic cultures appeared to express collagen I and collagen IV at significantly lower levels (assessed from staining intensity) than those seen in the two hypoxic cultures (FIG. 8). For laminin and fibronectin, there were no apparent distinctions in the intensity of stains in the four cultures. Laminin analysis showed no difference in their organization, which displayed a disorganized polymer network, whereas fibronectin formed fibrils as a result of the high cell densities. Fibronectin fibrils in all the cultures also showed increasing alignment as the cells grew to confluency.

### Cell Cycle and Viability

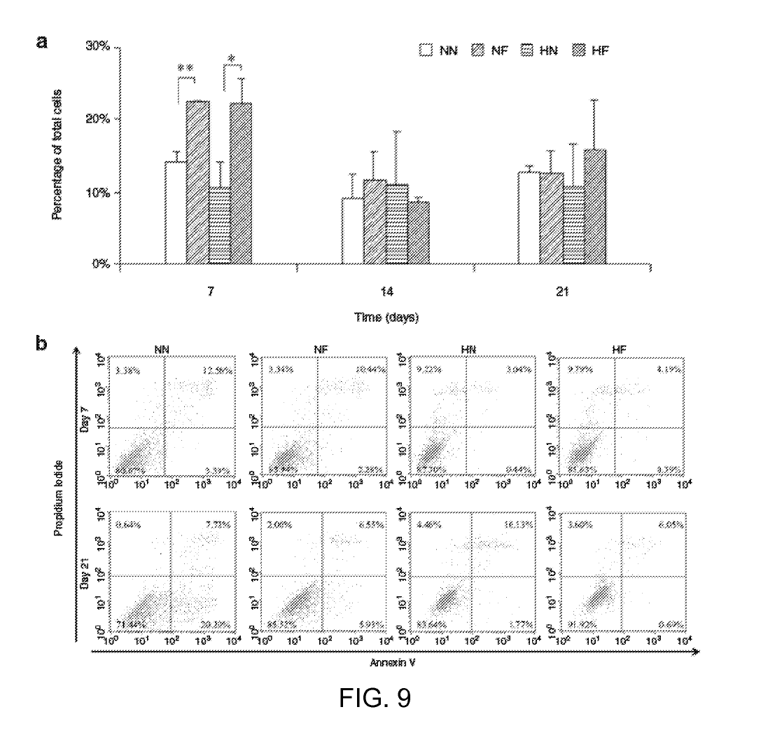

At day 7, the total percentage of cells in the proliferative state (S and M phases) on the two nanopatterned surfaces was significantly lower (p<0.05) than those on the two flat surfaces. Beyond day 7, no significant difference was detected when comparison was made between the types of substrates (FIG. 9A). Cell viability was evaluated by the flow cytometry-based Annexin V/PI apoptosis assay. In this assay, viable cells are both Annexin V and PI negative and cells undergoing apoptosis are Annexin V positive and PI negative, while dead cells are both Annexin V and PI positive. At day 7, a significantly higher percentage of dead cells were present in both NN and NF cultures (FIG. 9B). However, the percentage of necrotic and apoptotic cells was significantly lower than their counterparts on HN and HF contains, respectively. After 21 days of culture, the NN samples displayed the lowest percentage of viable cells (71.44%) and the highest percentage of apoptotic cells (20.20%), while the HF samples gave the highest percentage of viable cells (91.92%) and the lowest percentage of apoptotic cells (0.69%) (FIG. 9B). The percentage of apoptotic cells was 3.4 times higher in the NF (5.93%) than the HN (1.77%) samples.

### Colony Forming Unit-Fibroblasts (CFU-F) and Gene Expression

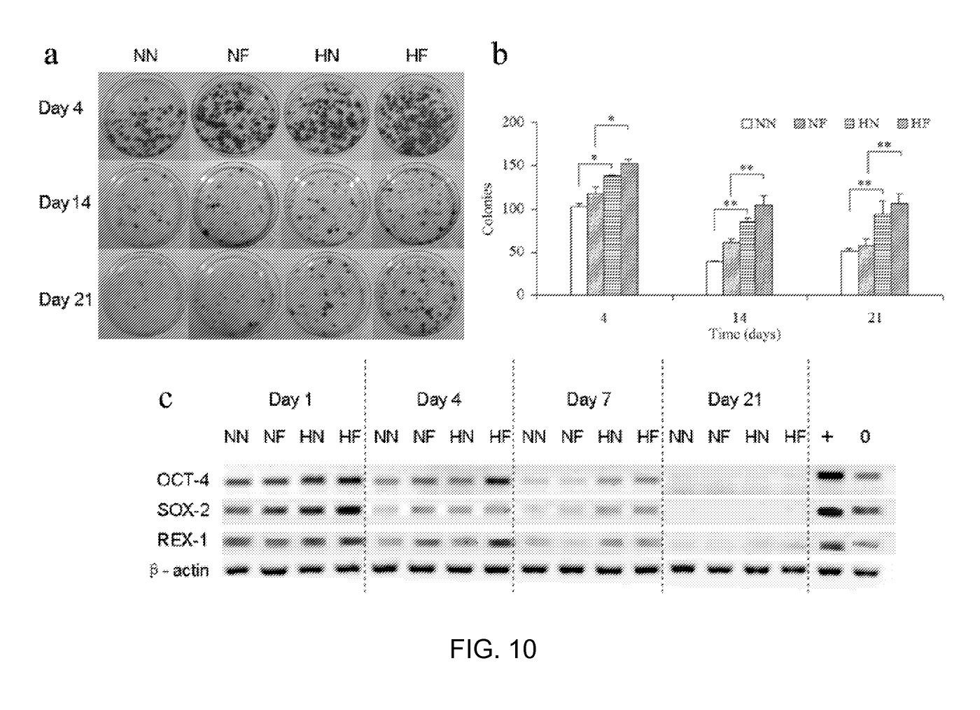

The proportion of primitive cells in the constructs was determined by measuring the colony-forming ability of the cells (FIG. 10A). All culture conditions resulted in a progressive reduction of progenitor population with time (FIG. 10B). At all time points there was no significant difference in colony numbers between the two normoxic cultures and the two hypoxic samples. However, a significant difference appeared between the hypoxic and normoxic cultures starting from day 4, with 1.5 to 1.8 fold higher progenitor cells observed at hypoxic compared to normoxic conditions (p<0.05).

To assess the sternness of hMSCs we analyzed three stem cell markers that correlate with pluripotency: Oct-4, Rex-1 and Sox-2, using the WiCELL hES cell line (H-9) as a control. Kolf, et al. (2007) Mesenchymal stromal cells—Biology of adult mesenchymal stem cells: regulation of niche, self-renewal and differentiation. Arthritis Res & Ther 9:204. RT PCR analysis showed that hypoxic conditions maintained a higher expression of all these stem cell markers in hMSCs grown on both nanopatterned and flat surfaces in the first 7 days compared to their normoxic counterparts (FIG. 10C). The expression levels in all samples decreased with time and were barely (Rex-1) or not (Oct-4 and Sox-2) detectable after 14 days.

### Differentiation

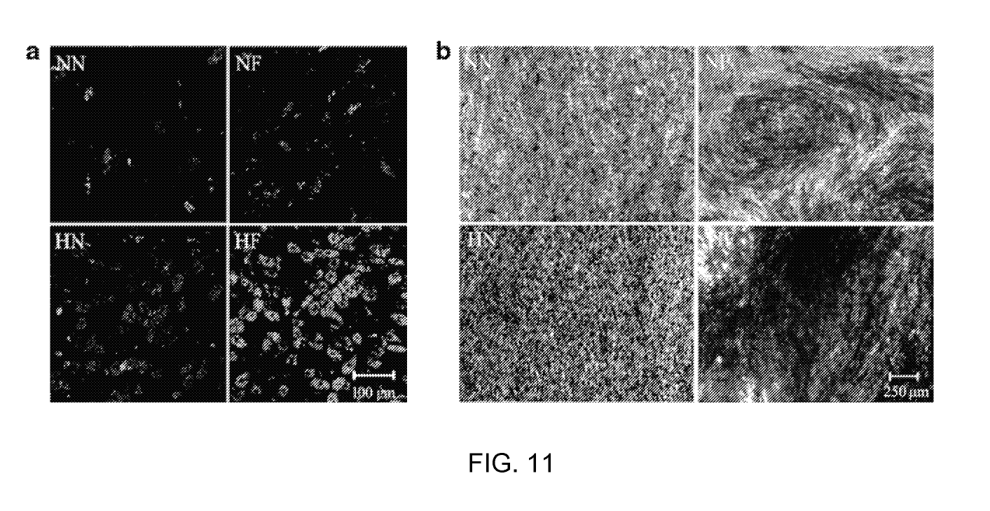

To determine if the cells still maintained their multi-lineage capabilities after extended periods of culture, differentiation assays were carried out on cells cultured under the four conditions. The cells from all experimental conditions displayed ability to differentiate along both osteoblastic and adipogenic lineages. Cells cultured under hypoxic conditions (HN and HF) showed more advanced adipogenesis with significantly more lipid vacuole formation than the normoxic counterparts (NN and NF) (FIG. 11A). Von Kossa assay also showed more advanced osteogenesis with darker mineralized deposits (FIG. 11B). On the nanopatterned surfaces, the cells from the hypoxic condition (HN) were still better aligned and with more calcium uniformly deposited than the normoxic counterpart (NN). On the flat surfaces, a larger and more uniform mineral deposited area was observed on the hypoxic sample (HF) than the normoxic one (NF).

### Discussion

The structural organization of tissues plays a critical role in dictating their function. Cell alignment is an important feature in many tissues. With the multipotency of hMSC, an aligned hMSC sheet would be a useful enabling technology for functional tissue engineering. To form an aligned hMSC sheet, the first crucial step is to grow hMSCs into confluency and high density, such that the cells can form tight junctions with each other, and secrete an abundance of ECM proteins to hold the cells together for harvesting. Topography is a potent cue to guide cell alignment. However, hMSCs tend to grow into a patchy structure (FIG. 6), and differentiate along neuronal, myogenic, and osteogenic pathways when cultured on synthetic nanostructures. For many tissue engineering applications it is highly desirable to maintain the undifferentiated state of the hMSCs so the cells can respond to the microenvironmental cues for optimal site-specific tissue development. Low O₂ tension can effectively support hMSC survival, maintain their primitive status and improve the secretion of ECM proteins.

### Cell Alignment and Uniformity on Synthetic Nanogratings

Substrate grating can effectively orient cells. Other than the width, the depth of the grating is also an important parameter. At the microscale, deep gratings appear to produce a non-uniform cell sheet. The portion of the cell layer grown on the ridges tends to be thinner, rendering the cell sheet more prone to tearing during handling and processing. Furthermore, deep grooves would likely lead to an increase in the time required for an intact sheet to form. We used a grating depth of 250 nm in this study, which appeared to be sufficient to orient the cells without causing heterogeneity of the cell sheet.

Low O₂ (2%) culture can considerably improve the uniformity of cell layers. Despite the high confluency at day 14, hMSCs were densely packed and uniformly aligned within the whole area, at the centimeter scale, when cultured under 2% O₂ (FIG. 6). The distribution of the cell nuclear alignment angles confirmed that the percentage of aligned cells was significantly higher in the 2% O₂ than the 20% O₂ condition (75% vs. 58%); and the range of alignment angles was also much narrower (30° vs. 90° with respect to the grating axis).

Connexin-43 is a gap junction protein secreted by hMSCs that plays an important role in cell-cell communication. It resides intracellularly and not at gap junctional plaques in the non-communicating cells. On the other hand, connexons are formed when the cells have uniform communications. In the 20% O₂ group, the connexin 43 protein was mostly confined within the cells on both nanopatterned and flat surfaces. In contrast, connexons, visible as bright fluorescence puncta, were uniformly distributed in the two low O₂ groups, indicating an established cell-cell communication in the cell layers.

The uniformity and high alignment of hMSCs grown on nanopatterned surfaces under low O₂ conditions are attributed to the significant secretion and more uniform distribution of the ECM protein fibronectin in the early stage (FIG. 7). Fibronectin contains cell adhesion domains that play an important role in promoting cell migration. Less secretion of fibronectin from hMSCs cultured under normal O₂ would result in weak cell-substrate interaction in the beginning and strong cell-cell adhesion at the later stage, the consequence of which is a higher degree of cell clustering (FIG. 7). This effect is even more acute on the nanopatterened surfaces. In contrast, when cultured under 2% O₂ the cells exhibited higher cell-substrate adhesion and improved motility, leading to uniform cell layers on both flat and nanopatterned PDMS surfaces.

In this study, the hMSCs grown under the same oxygen tension, regardless of whether the scaffold was nanopatterned or flat, exhibited similar nuclear shape with no significant difference in the morphology parameters of elongation and roundness (FIG. 6D). However, the nuclei of hMSCs from 2% O₂ condition displayed a rounder shape, whereas those from 20% O₂ were more elongated after 21 days culture. The difference in cell orientation and nuclear shape suggests that different physical forces are experienced by the cells between the two oxygen culture conditions.

The development of tissues and organs is guided by the organization and composition of ECM proteins. The physical state of the ECM, not only its molecular composition, functions as a regulator of cell-matrix adhesion. On vertical TiO₂ nanotubes where the ECM deposition is non-uniform, the hMSCs extend their filopodia to search for the ECM proteins, thus forming extraordinarily elongated shapes. We have also observed that during the first 2 days after cell seeding, the cells on nanopatterned surfaces under 2% O₂ were less spread than their normal oxygen counterparts. It is apparent that hMSCs grown under 2% O₂ secrete more fibronectin that reduce their need to reach for ECM protein aggregates. Thus, they are able to maintain a nucleus that is more spherical. There also appears to be significant differences in the level of collagen I and IV secreted by the cells under the two O₂ tensions. Transcriptional induction of collagen is proportional to the roundness of the nucleus. Thus, the nuclear elongation might affect the synthesis of collagen under normal oxygen tension, which eventually influences the hMSC phenotypes in the cell layer.

### Cell Viability, Sternness and Pluripotency

hMSCs possess great metabolic flexibility that enables them to survive in ischemic environments under hypoxia and glucose depletion, which may explain the lower percentage of apoptotic cells in the hypoxic samples at day 21. The higher expression of stem cell gene markers Oct-4, Rex-1 and Sox-2 in the hypoxic samples also suggests that the 2% O₂ condition is a more favorable microenvironment for hMSC self-renewal than under normoxic condition. The more advanced adipogenesis and osteogenesis observed in the differentiation assays for the hypoxic samples corroborates with the gene expression profiling and confirms the benefit of creating a hMSC layer under low O₂ tension for retention of a higher degree of sternness.

In summary, we report in this study the application of nanotopography and low O₂ (2%) tension culture conditions to create a confluent, well-aligned cell layer comprising hMSCs in a relatively undifferentiated state. The cell layers are more uniform and contain greater amount of ECM proteins than their counterparts from conventional cultures. With the well-preserved multi-lineage differentiation ability of the hMSCs, and coupled with other technologies that detach the cell layer from the substrate and create 3D structures as described above, this approach will expand the capability of cell sheet engineering for the regeneration of complex tissues.

1. A method of making a cell sheet comprising:
culturing cells with an at least partially coated nanoimprinted scaffold in 1%-10% oxygen conditions to form an aligned cell sheet; and
removing the aligned cell sheet from the coated scaffold.

2. The method of claim 1, wherein the scaffold is constructed from plastic, metal or ceramic.

3. The method of claim 1, wherein the scaffold is constructed of thermoplastic polymers.

4. The method of claim 1, wherein the scaffold is selected from the group consisting of poly(dimethylsiloxan) (PDMS), polystyrene, poly L-lactic acid, poly glycolic acid, poly hydroxybutyrate, polycarbonate (PC), polycaprolactone (PCL), polymethylmethacrylate (PMMA), and combinations thereof.

5. The method of claim 1, wherein the coating comprises chitosan, hydroxybutyl chitosan, collagen, fibronectin, laminin, elastin, fibrin, proteoglycans, gelatin, hyaluronan, or combinations thereof.

6. The method of claim 1, wherein the cells are selected from the group consisting of mesenchymal stem cells, myocyte precursor cells, myocytes, fibroblasts, chondrocytes, endothelial cells, epithelial cells, embryonic stem cells, hematopoetic stem cells, anchorage-dependent cell precursors, induced pluripotent stem cells (iPS cells), and combinations thereof.

7. The method of claim 6, wherein the induced pluripotent stem cells are selected from fibroblasts, keratinocytes, and mesenchymal stem cells.

8. The method of claim 1, wherein the cells are human mesenchymal stem cells.

9. The method of claim 7, wherein the cells remain pluripotent.

10. The method of claim 1, wherein the cells are cultured in 1% to 7% oxygen conditions.

11. The method of claim 1, wherein the cells are cultured in 1% to 3% oxygen conditions.

12. The method of claim 2, wherein the cells are cultured in 1% to 7% oxygen conditions.

13. The method of claim 12, wherein the cells are cultured in 1% to 3% oxygen conditions.

14. The method of claim 1, wherein the cells form tight junctions.

15. The method of claim 1, wherein at least 75% of the cells are aligned with an angle of less than 30° in the aligned cell sheet.

16. The method of claim 1, wherein at least 65% of the cells are aligned with an angle of less than 10° in the aligned cell sheet.

17. The method of claim 2, wherein at least 75% of the cells are aligned with an angle of less than 30° in the aligned cell sheet.

18. The method of claim 2, wherein at least 65% of the cells are aligned with an angle of less than 10° in the aligned cell sheet.

19. The method of claim 1, wherein the nanoimprinted scaffold has a nanograting with a depth of between 200 nm and 300 nm and a width of between about 300 nm and 400 nm.

20. The method of claim 2, wherein the nanoimprinted scaffold has a nanograting with a depth of between 200 nm and 300 nm and a width of between about 300 nm and 400 nm.

21. The method of claim 1, wherein the cell sheet is removed from the scaffold by dissolving the coating.

22. A cell sheet made by the method of claim 1.

23. A cell sheet comprising aligned cells, wherein at least 75% of the cells are aligned with an angle of less than 30° in the aligned cell sheet.

24. A cell sheet comprising aligned cells, wherein at least 65% of the cells are aligned with an angle of less than 10° in the aligned cell sheet.

25. A method of making a tissue sheet comprising stacking a plurality of the cell sheets of claim 22, and culturing in 1% to 10% oxygen to form a tissue sheet.

26. The method of claim 25, wherein 1% to 3% oxygen is used.

27. A tissue sheet made by the method of claim 25.

28. A tissue sheet comprising aligned cells, wherein at least 75% of the cells are aligned with an angle of less than 30° in the aligned tissue sheet.

29. A method of making a tissue-engineered blood vessel comprising wrapping at least one of the cell sheets of claim 22 around a mandrel to form a tube; culturing the tube in 1% to 5% oxygen; and removing the mandrel to form the tissue-engineered blood vessel.

30. A tissue-engineered blood vessel made by the method of claim 29.

31. A tissue-engineered blood vessel comprising aligned cells, wherein at least 75% of the cells are aligned with an angle of less than 30° in the tissue-engineered blood vessel.

32. A method of making skin comprising implanting the tissue sheet of claim 27 in the skin of a subject.

33. A method of making bone comprising implanting the tissue sheet of claim 27 in a subject in need of bone regeneration.

34. A method of treating a subject in need of a tissue graft comprising implanting the tissue sheet of claim 27 into the subject.

35. A method of making a blood vessel comprising implanting the tissue-engineered blood vessel of claim 30 in a subject.

36. A method of treating a vascular disease comprising implanting the tissue-engineered blood vessel of claim 30 into a subject with vascular disease.
